(19) United States
(12) Patent Application Publication
(10) Pub. No.: US 2009/0029937 A1
(43) Pub. Date: Jan. 29, 2009

(54) BIODEGRADABLE CATIONIC POLYMER GENE TRANSFER COMPOSITIONS AND METHODS OF USE

(75) Inventors: Chih-Chang Chu, Ithaca, NY (US); Martha A. Mutschler-Chu, Ithaca, NY (US); Hua Song, Ithaca, NY (US); Bo Liu, Roosevelt Island, NY (US); Zaza D. Gomurashvili, La Jolla, CA (US)

Correspondence Address: DLA PIPER LLP (US), 4365 EXECUTIVE DRIVE, SUITE 1100, SAN DIEGO, CA, 92121-2133, US

(73) Assignees: CORNELL UNIVERSITY, Ithaca, NY (US); MEDIVAS, LLC, San Diego, CA (US)

(21) Appl. No.: 12/177,682

(22) Filed: Jul. 22, 2008

Related U.S. Application Data
(60) Provisional application No. 60/961,876, filed on Jul. 24, 2007.

(51) Int. Cl. A61K 48/00; C12N 15/11; C12N 15/87; C12N 5/06; A61K 47/34; A61K 31/7105

(52) U.S. Cl. 514/44; 536/23.1; 435/455; 435/375; 514/772.3

(57) ABSTRACT

The invention provides biodegradable, cationic compositions based on cationic α-amino acid-containing PEA, PEUR and PEU polymers for use in preparation of non-viral gene transfer compositions. In the invention gene transfer compositions a poly nucleic acid is condensed with the polymer to form a soluble unit wherein the electrical charge of the poly nucleic acid is neutralized by the polymers. The invention gene transfer compositions can be used to transfect target cells by contact with the target cells.

### RELATED APPLICATIONS

This application claims priority under 35 U.S.C. §119(e) of U.S. provisional application Ser. No. 60/961,876, filed Jul. 24, 2007, which is hereby incorporated by reference in its entirety.

### BACKGROUND OF THE INVENTION

During the past decade, biodegradable, bioresorbable polymers for biomedical uses have garnered growing interest. Recently described, aliphatic PEAs based on α-amino acids, aliphatic diols, and fatty dicarboxylic acids have been found to be good candidates for biomedical uses because of their biocompatibility, low toxicity, and biodegradability (K. DeFife et al. Transcatheter Cardiovascular Therapeutics—TUT 2004 Conference. Poster presentation. Washington, D.C. 2004; G. Tsitlanadze, et al. J. Biomater. Sci. Polymer Edn. (2004). 15:1-24).

The highly versatile Active Polycondensation (APC) method, which is mainly carried out in solution at mild temperatures, allows synthesis of regular, linear, polyfunctional PEAs, poly(ester-urethanes) (PEURs) and poly(ester ureas) (PEUs) with high molecular weights. Due to the synthetic versatility of APC, a wide range of material properties can be achieved in these polymers by varying the three components—α-amino-acids, diols and dicarboxylic acids—used as building blocks to fabricate the macromolecular backbone; (R. Katsarava, et al. J. Polym. Sci. Part A: Polym. Chem. (1999) 37:391-407).

Gene therapy can be defined as the treatment of disease by the transfer of genetic material into specific cells of a subject. The concept of human gene therapy was first articulated in the early 1970s. Advances in molecular biology in the late 1970s and throughout the 1980s led to the first treatment of patients with gene-transfer techniques under approved FDA protocols in 1990. With optimistic results from these studies, gene therapy was expected to rapidly become commonplace for the treatment and cure of many human ailments. However, considering that 1131 gene-therapy clinical trials have been approved worldwide since 1989, the small number of successes is disappointing.

The genetic constructs used in gene therapy consist of three components: a gene that encodes a specific therapeutic protein; a plasmid-based gene expression system that controls the functioning of the gene within a target cell; and a gene transfer system that controls the delivery of the gene expression plasmids to specific locations within the body (Mahato, R. I. et al. Advances in Genetics (1999) 41:95-156). A key limitation to development of human gene therapy remains the lack of safe, efficient and controllable methods for gene transfer.

The use of viral vectors for human clinical use has historically encountered limitations, which may range from limited payload capacity and general production issues to immune and toxic reactions, as well as the potential for undesirable viral recombination. Polymers and lipids are the most common non-viral synthetic transfer vectors and have been developed in an effort to avoid the possibility of such limitations. Therefore, non-viral systems, especially synthetic DNA delivery systems, have become increasingly desirable in both research laboratories and clinical settings.

However, research in the field of non-viral gene transfer is in its infancy compared to research of viral-based gene transfer systems. Among the common cationic polymers that have been evaluated for this purpose, the best known are poly-L-lysine (PLL) and polyethylenimine (PEI). Other synthetic and natural polycations that have been developed as non-viral vectors include polyamidoamine dendrimers (Tomalia, D. A., et al. Angewandte Chemie-International Edition in English (1990) 29(2):138-175) and chitosan (Erbacher, P., et al. Pharmaceutical Research (1998) 15(9):1332-1339).

Polymers that have been specifically designed to improve gene transfer efficiency include imidazole-containing polymers with proton-sponge effect, membrane-disruptive peptides and polymers, such as polyethylacrylic acid (PEAA) and polypropylacrylic acid (PPAA); cyclodextrin-containing polymers and degradable polycations, such as poly[alpha-(4-aminobutyl)-L-glycolic acid] (PAGA) and poly(amino acid); and polycations linked to a nonionic water-soluble polymer, such as polyethylene oxide (PEO). In most cases, these polymers were designed to address a specific intracellular barrier, such as stability, biocompatibility or endosomal escape. The results have been mixed, with some polymers performing as well as, or even slightly better than, the best off-the-shelf polymers. However, none approach the efficiency of viruses as a gene transfer vector.

The above studies have shown that there are three major barriers to efficient DNA delivery: low uptake across the cell plasma membrane; inadequate release and instability of released DNA molecules, and difficulty of nuclear targeting. Thus, despite the above described advances in the art, there is a need for new and better non-viral gene transfer systems.

### SUMMARY OF THE INVENTION

Poly(ester-amide)s (PEAs) Poly(ester urethane)s PEURs and Poly(ester urea)s (PEUs) are a family of novel biodegradable polymers composed of both amide and either ester, urethane or urea blocks on their backbones. PEAs have been studied widely for many years because they combine the favorable properties of both polyesters and polyamides. Natural amino acids that are positively charged at biological pH were chosen as the resource for the amine group of the cationic PEAs, PEURs and PEUs used in the invention gene transfer compositions due to their natural abundance and biocompatibility. For example, L-arginine is an α-amino acid present in the proteins of all life forms. It carries a positive charge at physiological pH due to the strong basic guanidino group with a pKa value of about 12. The cationic groups present in α-amino acid containing PEAs and related PEURs and PEUs provide the basic character in the polymers used in the invention gene transfer compositions necessary for condensing nucleic acid sequences, such as DNA and RNA, which are negatively charged, into a soluble complex.

Accordingly, in one embodiment the invention provides a biodegradable gene transfer composition comprising at least one poly nucleic acid condensed into a soluble complex with a cationic polymer comprising at least one of the following:

a PEA polymer having a chemical formula described by general structural formula (I),

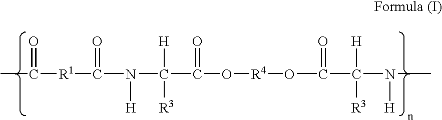

wherein n ranges from about 5 to about 100; R¹ is independently selected from (C₂-C₁₂) alkyl or alkenyl; R³s in individual n units are independently selected from the group consisting of (CH₂)₃NHC(═NH₂⁺)NH₂, 4-methylene imidazolinium, (CH₂)₄NH₃⁺, (CH₂)₃NH₃₄ and combinations thereof; and R⁴ is independently (C₂-C₅) alkyl;

or a poly(ester urethane) (PEUR) polymer having a chemical formula described by structural formula (II),

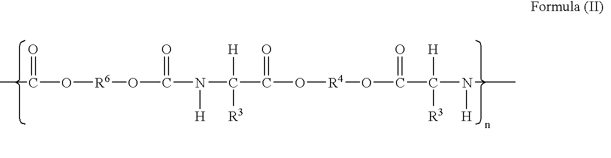

wherein n ranges from about 5 to about 100; R³s in individual n units are independently selected from the group consisting of (CH₂)₃NHC(═NH₂⁺)NH₂, (CH₂)₄NH₃⁺, (CH₂)₃NH₃⁺, 4-methylene imidazolinium, and combinations thereof; and R⁴ and R⁶ are independently (C₂-C₅) alkyl;

or a poly(ester urea) (PEU) having a chemical formula described by general structural formula (III):

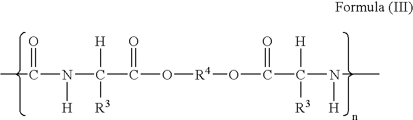

wherein n ranges from about 5 to about 100; R³s in individual n units are independently selected from the group consisting of (CH₂)₃NHC(═NH₂⁺)NH₂, (CH₂)₄NH₃⁺, (CH₂)₃NH₃⁺, 4-methylene imidazolinium, and combinations thereof; and R⁴ is independently (C₂-C₅) alkyl.

In another embodiment, the invention provides methods for transfecting a target cell by incubating the target cell with the invention gene transfer composition so as to transfect the target cell.

### A BRIEF DESCRIPTION OF THE FIGURES

FIG. 1 is a chemical reaction scheme showing three steps in synthesis of arginine-based poly(ester amides) (Arg-PEAs).

FIG. 2 is a graph showing the effect of the amount of various polymers on the ability of the polymer to condense DNA into a soluble complex as monitored by ethidium bromide displacement assay.

FIG. 3 is a graph showing the effect of hydrophobic block length of various Arg-PEAs on the ability of the cationic polymer to condense plasmid DNA into a soluble complex as monitored by the ethidium bromide displacement assay. PEI and PLL polymers are controls.

FIG. 4 is a graph showing the effect of different counter ions on the ability of Arg-PEA polymers to condense DNA in different salt formations. Toluenesulfonic salt ═S and hydrochloride salt ═Cl.

FIG. 5 is a bar graph illustrating efficiency of various condensed polymer/DNA plasmid complexes for transfecting Vascular Smooth Muscle Cells (SMCs) as measured by expression and luminescence (RLI) therein of firefly luciferase by transfected cells. Plasmid DNAs used were COL(-772)/Luc and pRL-CMV (10:1 w/w). SUPERFECT® (SF), polyethylenimine (PEI) and poly-L-lysine (PLL) were tested at the known optimum weight ratio of polymer to DNA. Various weight ratios of plasmid DNA to 2-Arg-3-S PEA polymer (PEA) were as shown in parentheses.

FIG. 6 is a bar graph showing transfection efficiency of cationic polymer: plasmid DNA complexes as measured by expressed renilla luciferase activity. Plasmid DNAs used were COL (-772)/Luc and pRL-CMV (10:1 w/w). SUPERFECT® (SF), PEI and PLL were tested using the known optimum weight ratio to DNA. Various weight ratios of 2-Arg-3-S PEA polymer:plasmid DNA tested were as shown in parentheses.

FIG. 7 is a bar graph showing the transfection efficiency of polymer/DNA complexes as measured by expressed ratio of firefly luciferase activity to renilla luciferase activity in transfected SMCs. Plasmid DNAs used were COL(-772)/Luc and pRL-CMV (10:1 w/w). SUPERFECT® (SF), PEI and PLL were tested using the known optimum weight ratio of polymer to DNA. Various weight ratios of 2-Arg-3-S PEA polymer:plasmid DNA were as shown in parentheses.

FIG. 8 is a bar graph showing the results of an MTT viability assay of Vascular SMCs transfected with invention gene transfer compositions at the indicated polymer:DNA weight ratios (in parenthesis). Control=cells only (without polymer); SF=SUPERFECT®; PEA=2-Arg-3-S. Bars in black indicate the corresponding polymer to DNA weight ratio for optimum transfection efficiency.

FIG. 9 is a graph showing cytotoxicity of polymer/DNA complex by MTT assay using spectrophotometric absorbances at 570 nm. Control=cells only (without polymer treatment). Numerals in parentheses=weight ratios of polymer to DNA tested on Superfect, PLL and PEA=(2-Arg-3-S). Bars in black=polymer to DNA weight ratio at optimum transfection efficiency.

### A DETAILED DESCRIPTION OF THE INVENTION

The present invention is based on the discovery that polymers that contain at least two positively charged natural α-amino acids per repeat unit at physiological pH, such as the poly (ester amides) PEAs, poly(ester urethanes) PEURs and poly (ester ureas) (PEUs) described herein, can be used to condense and deliver poly nucleic acids into target cells for various in vivo applications, such as in gene therapy.

The ability of the PEAs, PEURs and PEUs used in the invention gene transfer compositions to condense poly nucleic acids results, at least in part, from the polymer repeat units containing two positively charged α-amino acids, as is illustrated in the Examples herein by PEAs that contain two L-Arginines per repeat unit. Prior to protonation, Arginine has one major and two less significant resonance contributors. However, protonated arginine has three significant resonance contributors, thereby stabilizing the protonated Arginine cation, an electrical configuration that provides the basic character in the polymer to condense poly nucleic acids placed in contact therewith.

Accordingly, the present invention provides gene transfer compositions comprising at least one poly nucleic acid condensed into a soluble complex with a cationic polymer comprising at least one or a blend of: a poly(ester amide) (PEA), poly(ester urethane) (PEUR) or poly(ester urea) (PEU) containing at least two positively charged natural α-amino acids per repeat unit. The invention gene transfer compositions can be soluble in water and other aqueous conditions, for example, under biological conditions, such as in blood, and the like, or in water/alcohol mixtures.

For biocompatibility, the PEA, PEUR and PEU polymers in the invention delivery systems were designed to contain hydrophilic residues of nontoxic, naturally occurring components or their derivatives-short aliphatic diols and di-acids and hydrophilic, positively charged α-amino acids.

More particularly, the building blocks of the repeat units of the PEA, PEUR and PEU polymers are composed of residues of short aliphatic diols and di-acids and hydrophilic L or D α-amino acids that are positively charged (such as arginine, ornithine, histidine, and lysine). Hydrophilicity of these aliphatic PEA, PEUR and PEU polymers can be varied and controlled by judicious selection of the hydrophilicity of the building blocks from which the polymers are derived, which hydrophilicities are well known in the art and as described herein.

More particularly, in one embodiment the invention provides a gene transfer composition comprising at least one poly nucleic acid condensed into a soluble complex with a cationic polymer comprising at least one of the following:

a PEA polymer having a chemical formula described by general structural formula (I),

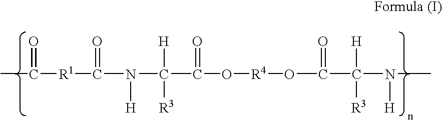

wherein n ranges from about 5 to about 100; R¹ is independently selected from (C₂-C₅) alkyl or alkenyl; R³s in individual n units are independently selected from the group consisting of (CH₂)₃NHC(═NH₂⁺)NH₂, 4-methylene imidazolinium, (CH₂)₄NH₃⁺, (CH₂)₃NH₃⁺ and combinations thereof; and R⁴ is independently (C₂-C₅) alkyl;

or a poly(ester urethane) (PEUR) polymer having a chemical formula described by structural formula (II),

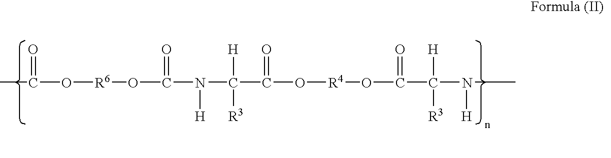

wherein n ranges from about 5 to about 100; R³s in individual n units are independently selected from the group consisting of (CH₂)₃NHC(═NH₂⁺)NH₂, (CH₂)₄NH₃⁺, (CH₂)₃NH₃⁺, 4-methylene imidazolinium, and combinations thereof; and R⁴ and R⁶ are independently (C₂-C₁₂) alkyl;

or a PEU having a chemical formula described by general structural formula (III):

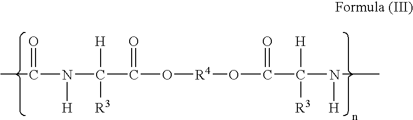

wherein n ranges from about 5 to about 100; R³s in individual n units are independently selected from the group consisting of (CH₂)₃NHC(═NH₂⁺)NH₂, (CH₂)₄NH₃⁺, (CH₂)₃NH₃⁺, 4-methylene imidazolinium, and combinations thereof; and R⁴ is independently (C₂-C₅) alkyl.

The structural formula for 4-methylene imidazlionium is as follows:

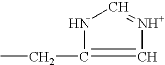

In certain embodiments, in addition to the cationic α-amino acids contained in the polymers used in this invention, presently preferred residues of aliphatic diols and di-acids for incorporation into the invention polymers are residues of two or three carbon diols and of two or three carbon aliphatic dicarboxylic acids (e.g., succinic and glutaric acids). The shorter the aliphatic segments in the backbone of the polymer compositions, the more water soluble the polymer will be and the greater will be the charge density of individual monomer units.

In certain additional embodiments, the polymer(s) in the composition can have one or more counter-ions associated with positively charged groups therein and/or one or more protecting groups bound to the polymer. Known examples of counter-ions suitable to associate with the polymer in the invention composition are counter-anions of weak acids having a pKa from about −7 to +5. Examples of such counter-anions include Cl⁻, F⁻, Bra, CH₃COO⁻, CF₃COO⁻, CCl₃COO⁻, and TosO⁻.

As used herein, the terms “water solubility” and “water soluble” as applied to the invention gene transfer compositions means the concentration of the composition per milliliter of deionized water at the saturation point of the composition therein. Water solubility will be different for each different polymer, but is determined by the balance of intermolecular forces between the solvent and solute and the entropy change that accompanies the solvation. Factors such as pH, temperature and pressure will alter this balance, thus changing the solubility. The solubility is also pH, temperature, and pressure dependent.

As generally defined, water soluble polymers can include truly soluble polymers to hydrogels (G. Swift, Polymer Degr. Stab. 59: (1998) 19-24). Invention compositions can be scarcely soluble (e.g., from about 0.01 mg/mL), or can be hygroscopic and when exposed to a humid atmosphere can take up water quickly to finally form a viscous solution in which composition/water ratio in solution can be varied infinitely.

The solubility of the polymers used in invention gene transfer compositions in deionized water at atmospheric pressure is in the range from about 0.01 mg/mL to 400 mg/mL at a temperature in the range from about 18° C. to about 55° C., preferably from about 22° C. to about 40° C. Quantitative solubility of the invention compositions can be visually estimated according to the method of Braun (D. Braun et al. in Praktikum der Makromolekularen Organischen Chemie, Alfred Huthig, Heidelberg, Germany, 1966). As is known to those of skill in the art, the Flory-Huggins solution theory is a theoretical model describing the solubility of polymers. The Hansen Solubility Parameters and the Hildebrand solubility parameters are empirical methods for the prediction of solubility. It is also possible to predict solubility from other physical constants, such as the enthalpy of fusion.

The addition of a low molecular weight electrolyte to a solution of a PEA, PEUR or PEUR polymer as described herein in deionized water can induce one of four responses. The electrolyte can cause chain contraction, chain expansion, aggregation through chelation (conformational transition), or precipitation (phase separation). The exact nature of the response will depend on various factors, such as the chemical structure, concentration, and molecular weight of the polymer and nature of added electrolyte. Nevertheless, invention gene transfer compositions can be soluble in various aqueous conditions, including those found in physiological conditions, such as blood, serum, tissue, and the like, or in water/alcohol solvent systems.

The water solubility of the invention compositions can also be characterized using such assays as static light scattering and size exclusion chromatography (SEC). Additionally, polymers can be characterized by ¹H NMR, ¹³C NMR, gel permeation chromatography (GPC), and differential scanning calorimetry (DSC), as is known in the art and as illustrated in the Examples herein.

All amino acids can exist as charged species, because of the terminal amino and carboxylate groups, but only a subset of amino acids have side chains that can, under suitable conditions, be charged. An amino residue is what remains after polymerization of an amino acid monomer into a polymer, such as a PEA, PEUR or PEU as described herein, and R³ in Formulas (I, II and III) refers to the pendant side chain of such an amino acid residue.

The term “cationic α-amino acid” as used herein to describe the invention polymers, means the R³ groups therein are those of amino acid residues whose side chains can function as weak acids—those not completely ionized when dissolved in water. The ionizable property is conferred upon these R³ groups by the presence therein of an ionizable moiety consisting of a proton that is covalently bonded to a heteroatom, such as an oxygen, sulfur or nitrogen. Under suitable aqueous conditions, such as the proximity of another ionizable molecule or group, the ionizable proton dissociates from R³ as the donating hydrogen ion, rendering R³ a base which can, in turn, accept a hydrogen ion. Dissociation of the proton from the acid form, or its acceptance by the base form is strongly dependent upon the pH of the aqueous milieu. Ionization degree is also environmentally sensitive, being dependent upon the temperature and ionic strength of the aqueous milieu as well as upon the micro-environment of the ionizable group within the polymer.

Thus, the term “cationic α-amino acid”, as used herein to describe certain of the polymers in invention gene transfer compositions, means the R³ groups of amino acid residues therein can form positive ions under suitable ambient aqueous or solvent conditions, especially under physiological conditions, such as in blood and tissue. Counter-ions of such positive amino acids can be as described above.

As used herein, the term “residue of a di-acid” means that portion of a dicarboxylic-acid that excludes the two carboxyl groups of the di-acid, which portion is incorporated into the backbone of the invention polymer compositions. As used herein, the term “residue of a diol” means that portion of a diol that excludes the two hydroxyl groups thereof at the points the residue is incorporated into the backbone of the invention polymer compositions. The corresponding di-acid or diol containing the “residue” thereof is used in synthesis of the invention gene transfer compositions.

The di-aryl sulfonic acid salts of diesters of α-amino acid and diol can be prepared by admixing α-amino acid, e.g., p-aryl sulfonic acid monohydrate, and diol in toluene, heating to reflux temperature, until water evolution has ceased, then cooling.

Saturated di-p-nitrophenyl esters of dicarboxylic acid and saturated di-p-toluene sulfonic acid salts of bis-α-amino acid esters can be prepared as described in U.S. Pat. No. 6,503,538 B1.

PEA, PEUR and PEU polymers of Formulas (I-III) containing cationic α-amino acids, as described herein, can be prepared using protective group chemistry. Protected monomers will be de-protected either prior to APC or after polymer work-up. Suitable protective reagents and reaction conditions used in protective group chemistry can be found, e.g. in Protective Groups in Organic Chemistry, Third Edition, Greene and Wuts, Wiley & Sons, Inc. (1999), the content of which is incorporated herein by reference in its entirety.

The poly nucleic acid in the invention compositions, as the term is used herein, can include deoxyribonucleic acid (DNA), ribonucleic acid (RNA), double stranded DNA, double stranded RNA, duplex DNA/RNA, antisense poly nucleic acids, functional RNA or a combination thereof. In one embodiment, the poly nucleic acid can be RNA. In another embodiment, the poly nucleic acid can be DNA. In another embodiment, the poly nucleic acid can be an antisense poly nucleic acid. In another embodiment the poly nucleic acid can be a sense poly nucleic acid. In another embodiment, the poly nucleic acid can include at least one nucleotide analog. In another embodiment, the poly nucleic acid can include a phosphodiester linked 3′-5′ and 5′-3′ poly nucleic acid backbone. Alternatively, the poly nucleic acid can include non-phosphodiester conjugations, such as phosphotioate type, phosphoramidate and peptide-nucleotide backbones. In another embodiment, moieties can be linked to the backbone sugars of the poly nucleic acid. Methods of creating such conjugations are well known to those of skill in the art.

The poly nucleic acid can be a single-stranded poly nucleic acid or a double-stranded poly nucleic acid. The poly nucleic acid can have any suitable length. Specifically, the poly nucleic acid can be about 2 to about 5,000 nucleotides in length, inclusive; about 2 to about 1000 nucleotides in length, inclusive; about 2 to about 100 nucleotides in length, inclusive; or about 2 to about 10 nucleotides in length, inclusive.

An antisense poly nucleic acid is typically a poly nucleic acid that is complimentary to an mRNA that encodes a target protein. For example, the mRNA can encode a cancer promoting protein i.e., the product of an oncogene. The antisense poly nucleic acid is complimentary to the single-stranded mRNA and will form a duplex and thereby inhibit expression of the target gene, i.e., will inhibit expression of the oncogene. The antisense poly nucleic acids of the invention can form a duplex with the mRNA encoding a target protein and will disallow expression of the target protein.

The term “functional RNA”, as used herein, refers to a ribozyme or other RNA that is not translated.

The term “poly nucleic acid decoy”, as used herein refers to a poly nucleic acid that inhibits the activity of a cellular factor upon binding of the cellular factor to the poly nucleic acid decoy. The poly nucleic acid decoy contains the binding site for the cellular factor. Examples of such cellular factors include, but are not limited to, transcription factors, polymerases and ribosomes. An example of a poly nucleic acid decoy for use as a transcription factor decoy will be a double-stranded poly nucleic acid containing the binding site for the transcription factor. Alternatively, the poly nucleic acid decoy for a transcription factor can be a single-stranded nucleic acid that hybridizes to itself to form a snap-back duplex containing the binding site for the target transcription factor. An example of a transcription factor decoy is the E2F decoy. E2F plays a role in transcription of genes that are involved with cell-cycle regulation and that cause cells to proliferate. Controlling E2F allows regulation of cellular proliferation. For example, after injury (e.g., angioplasty, surgery, stenting) smooth muscle cells proliferate in response to the injury. Proliferation may cause restenosis of the treated area (closure of an artery through cellular proliferation). Therefore, modulation of E2F activity allows control of cell proliferation and can be used to decrease proliferation and avoid closure of an artery. Examples of other such poly nucleic acid decoys and target proteins include, but are not limited to, promoter sequences for inhibiting polymerases and ribosome binding sequences for inhibiting ribosomes. It is understood that the invention includes poly nucleic acid decoys constructed to inhibit any target cellular factor.

The term “gene therapy agent”, as used herein, refers to an agent that causes expression of a gene product in a target cell through introduction of a gene into the target cell followed by expression of the gene product. An example of such a gene therapy agent would be a genetic construct that causes expression of a protein when introduced into a cell, such as a DNA vector. Alternatively, a gene therapy agent can decrease expression of a gene in a target cell. An example of such a gene therapy agent would be the introduction of a poly nucleic acid segment into a cell that would integrate into a target gene or otherwise disrupt expression of the gene. Examples of such agents include poly nucleic acids that are able to disrupt a gene through homologous recombination. Methods of introducing and disrupting genes within cells are well known to those of skill in the art and as described herein.

In one embodiment, the poly nucleic acid can be synthesized according to commonly known chemical methods. In another embodiment, the poly nucleic acid can be obtained from a commercial supplier. The poly nucleic acid can include, but is not limited to, at least one nucleotide analog, such as bromo derivatives, azido derivatives, fluorescent derivatives and combinations thereof. Nucleotide analogs are well known to those of skill in the art. The poly nucleic acid can include a chain terminator. The poly nucleic acid can also be used, e.g., as a cross-linking reagent or a fluorescent tag. Many common conjugations can be employed to couple a poly nucleic acid to another moiety, e.g., phosphate, hydroxyl, etc. Additionally, a moiety may be linked to the poly nucleic acid through a nucleotide analog incorporated into the poly nucleic acid. In another embodiment, the poly nucleic acid can include a phosphodiester linked 3′-5′ and 5′-3′ poly nucleic acid backbone. Alternatively, the poly nucleic acid can include non-phosphodiester conjugations, such as phosphothioate type, phosphoramidate and peptide-nucleotide backbones. In another embodiment, moieties can be linked to the backbone sugars of the poly nucleic acid. Methods of creating such conjugations are well known to those of skill in the art.

The condensed polymer/poly nucleic acid can degrade in vitro in the presence of an enzyme, such as α-chymotrypsin, or when injected in vivo to provide time release of a suitable and effective amount of the poly nucleic acid. Typically, the suitable and effective amount of poly nucleic acid can be released in a time range from about twenty-four hours to about seven days. Any suitable and effective period of time can be chosen by judicious selection of certain factors. Factors that typically affect the length of time over which the poly nucleic acid is released from the invention composition include, e.g., the nature and amount of polymer, the nature, size and amount of poly nucleic acid, the pH, and the temperature and electrolyte or enzyme content of the environment into which the composition is introduced.

Any suitable size of PEA, PEUR or PEU polymer of Formula (I, II or III) can be employed in the invention gene deliver compositions. For example, the polymer can have a size within the range from about 1×10⁻⁹ meters to about 1×10⁻⁶ meters.

The invention gene transfer compositions and methods of use described herein encompass the use and delivery to target cells of poly nucleic acids, including any type of RNA or DNA. “DNA”, as the term is used herein, encompasses a plasmid for expression of a gene contained therein, such as a gene encoding a therapeutic molecule. The term “RNA”, as used herein encompasses messenger (mRNA), transfer (tRNA), ribosomal (rRNA), and interfering (iRNA). Interfering RNA is any RNA involved in post-transcriptional gene silencing, which definition includes, but is not limited to, double stranded RNA (dsRNA), small interfering RNA (siRNA), and microRNA (miRNA) that are comprised of sense and antisense strands. In the mechanism of RNA interference, dsRNA enters a cell and is digested to 21-23 nucleotide siRNAs by the enzyme DICER therein. Successive cleavage events degrade the RNA to 19-21 nucleotides known as siRNA. The siRNA antisense strand binds a nuclease complex to form the RNA-induced silencing complex, or RISC. Activated RISC targets the homologous transcript by base pairing interactions and cleaves the mRNA, thereby suppressing expression of the target gene. Recent evidence suggests that the machinery is largely identical for miRNA (Cullen, B. R. (2004) Virus Res. 102:3). In this way, iRNA, once condensed with the polymer, can be delivered into a cell by phago- or pino-cytosis and released to enter the cell's normal biological processing pathway as a means of suppressing expression of a target gene.

The emerging sequence-specific inhibitors of gene expression, small interfering RNAs (siRNAs), have great therapeutic potential; however, development of such molecules as therapeutic agents is hampered by rapid degradation of siRNA in vivo. Therefore a key requirement for success in therapeutic use of siRNA is the protection of the gene silencing poly nucleic acid. In the present invention, such protection for siRNA is provided by condensation of the poly nucleic acid molecule with the cationic PEA, PEUR or PEU polymers described herein.

For, example, in fabrication of the invention composition for delivery of the antisense strand of iRNA, the antisense strand of negatively charged iRNA is condensed with the cationic polymer. The dsRNA is condensed with the carrier polymer. Alternatively, the sense strand can be condensed with one polymer chain and the antisense strand with another polymer chain. In either case, double stranded RNA is released from the invention composition during biodegradation of the polymer, and the antisense strand, freed from the sense strand, would enter the normal biological pathway for iRNA.

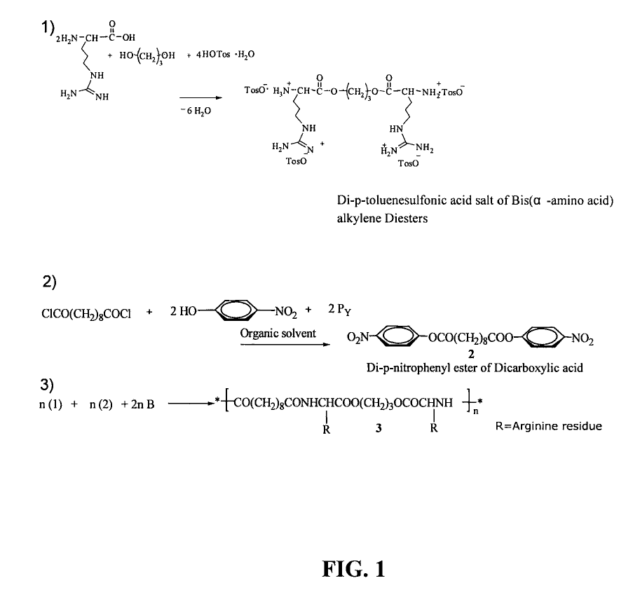

To illustrate the invention gene transfer compositions, a group of positively charged water soluble Arginine based Poly(Ester-Amide)s (Arg-PEAs) were synthesized by solution polycondensation of two monomers, di-p-Nitrophenyl esters of di-acids: succinic, adipic or sebacic acids (NSu, NA, or NS) and bis(L-Arginine)-diol diester di-p-toluenesulfonate salts (Arg2, Arg3, or Arg4) according to the reaction scheme shown in FIG. 1. The general chemical structure of Arg-PEAs is shown in structural formula (IV) below,

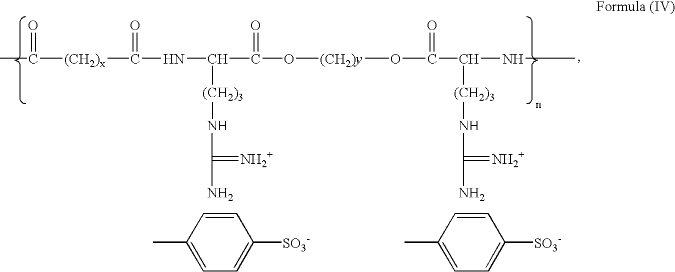

wherein the polymers are named using the general convention x-Arg-y-S, wherein x is the number of methylene groups between two closest ester groups and y is the number of methylene groups between two closest amide groups. The hydrophobicity and positive charge density of the polymers can be varied by changing x and y (as shown in Table 1).

There are two Arginines in every repeat unit of the Arg-PEA of structural formula (I), and therefore two positive charges are counted for every repeat unit. It is then rational to assume that the shorter the repeat unit (the smaller x or y) or the lighter the formula weight of the repeat unit, the higher the charge density. For example, among the tested Arg-PEAs (Table 2), 8-Arg-4-S has the most hydrophobicity and least charge density, while 2-Arg-2-S has the least hydrophobicity and the greatest charge density. By comparison, the formula weight of a repeat unit of comparison control polymer PEI is 43, while that of 2-Arg-2-S is 800.9, about 19 times that of PEI. Therefore, PEI obviously has a much higher charge density than the Arg-PEAs.

The guanidine group of L-Arginine is such a strong base that the p-toluenesulfonic acid, which is usually removed by triethylamine in the polycondensation step in the case of hydrophobic amino acid PEA synthesis, remains tightly attached to an Arg-PEA. As a result, negatively charged DNA needs to compete with p-toluenesulfonic acid first in order to interact with the guanidine group. Winning of the competition by the DNA is not always expected, so a large amount of the Arg-PEA polymer is required to fully interact with and condense a given amount of DNA.

TABLE 1
L-Arginine based PEAs, PEI and PLL: naming and chemical formula.

| | | | Empirical | |
|---|---|---|---|---|
| N | Polymer | Naming | Formula | Formula weight |
| 1 | | 2-Arg-2-S | C₃₂H₄₈N₈O₁₂S₂ | 800.9 |
| 2 | | 2-Arg-3-S | C₃₃H₅₀N₈O₁₂S₂ | 814.9 |
| 3 | Poly(ester- | 4-Arg-3-S | C₃₅H₅₄N₈O₁₂S₂ | 843.0 |
| | amide) | | | |
| 4 | x-Arg-y-S | 4-Arg-4-S | C₃₆H₅₆N₈O₁₂S₂ | 857.0 |
| 5 | | 8-Arg-3-S | C₃₉H₆₂N₈O₁₂S₂ | 899.1 |
| 6 | | 8-Arg-4-S | C₄₀H₆₄N₈O₁₂S₂ | 913.1 |
| 7 | Poly(ester- | 2-Arg-2-Cl | C₁₈H₃₅N₈O₆C1₂ | 529.5 |
| | amide) | | | |
| 8 | x-Arg-y-Cl | 4-Arg-3-Cl | C₁₈H₃₅N₈O₆Cl₂ | 571.5 |
| 9 | PEI | PEI | C₂H₅N | 43.0 |
| 10 | PLL | PLL | C₆H₁₄OBr | 209.0 |

Physico-chemical tests (gel electrophoresis, fluorescence, and luciferase expression assays) confirmed that Arginine based PEA polymers condensed plasmid DNA sufficiently for the invention gene transfer compositions to easily enter vascular smooth muscle cells in vitro. Luciferase expression assays were performed to evaluate transfection efficiency of the invention gene transfer compositions as compared with commercial gene transfer agent SUPERFECT® and the most efficient known polymer gene transfer agents PLL and PEI. The Arg-PEAs tested showed about 50% of the transfection efficiency of SUPERFECT®, with much lower cytotoxicity and a 5-10 fold increase in transfection efficiency over that of PLL and PEI.

Studies were also conducted to examine the effect of chemical structure on properties of the Arg-PEA polymers that are related to their use as a gene transfer vector. For example, it was discovered that increasing hydrophobicity or increasing the number of methylene groups in the polymer backbone (i.e., increasing the distance) between the charge centers in the cationic PEA, PEUR and PEU polymers used in the invention compositions and methods will decrease the ability of the polymer to condense plasmid DNA, and the longer the distance is between charge centers, the greater the decrease is. However, when the distance between positive charge centers is at a minimum (e.g., two methylene groups), both steric hindrance and the effect of negatively charged counter-ions on the ability of the polymer to condense DNA play a larger role. The greater the negative pKa of the acid from which the counter-ion comes, the greater is the deterrent effect of the counter-ion on the ability of the polymer to condense DNA.

The L-Arginine based poly(ester-amide)s synthesized by solution polycondensation as described herein (see Example 1) were evaluated for efficiency as a non-viral gene carrier to effect transfection of a target cell, for example to be used in gene therapy. Gel retardation and ethidium bromide displacement assays were used to confirm that the positively charged PEA polymers were able to neutralize negatively charged plasmid DNA to form a compact complex suitable for use in transfection of a target cell and for transgenic production of a heterologous protein in cells transfected with the invention gene transfer compositions. As shown in Example 2 herein, Collagen-Luciferase and PRL-CMV were expressed in vitro as reporter genes by vascular smooth muscle cells (SMCs) transfected using invention gene transfer compositions. In vitro transfection efficiency and cytotoxicity of Arg-PEA polymers were measured by luciferase activity reading and MTT (3-(4,5-Dimethylthiazol-2-yl)-2,5-diphenyltetrazolium bromide) assay. Commercially available gene transfer reagent SUPERFECT® and the most recognized non-viral gene transfer polymers, PEI and PLL, were used as comparison controls.

Tests designed to discover the optimum relationships between polymer structure and physical properties of Arg-PEAs studied the effect of the length of repeat unit on hydrophobicity and the effect of steric hindrance caused by counter-ions with the polymers. The highest transfection efficiency among the Arg-PEA polymers tested was achieved by Arg-PEAs with either the shortest repeat unit or highest charge density at a polymer:DNA weight ratio of 5900:1. Tests comparing the transfection efficiency of Arg-PEA with PLL, PEI and SUPERFECT® as a gene transfer vector (Example 2) showed that Arg-PEA was about 10-fold more efficient than PLL and PEI, and about 40% as efficient as the commercial transfection reagent, SUPERFECT®.

The following Examples are meant to illustrate, and not to limit, the invention.

### EXAMPLE 1

### Synthesis and Characterization of Positively Charged Water Soluble Poly(Ester Amide)s

Materials: L-Arginine (L-Arg), p-toluenesulfonic acid monohydrate, sebacoyl chloride, adipoyl chloride, ethylene glycol, 1,3-propanediol, 1,4-butanediol (Alfa Aesar, Ward Hill, Mass.) and p-nitrophenol (J. T. Baker, Phillipsburg, N.J.) were used without further purification. Triethylamine (Fisher Scientific, Fairlawn, N.J.) was dried by refluxing with calcium hydride, and then distilled. Solvents such as toluene, ethyl acetate, acetone, 2-propanol and dimethyl sulfoxide (DMSO) were purchased from VWR Scientific (West Chester, Pa.) and were purified by standard methods before use.

Synthesis of monomers and polymers: The general scheme used in synthesis of PEAs was adapted for synthesis of Arg-PEAs (FIG. 1): the preparation of di-p-toluenesulfonic acid salts of bis(L-arginine)-α,ω-alkylene diesters (1), the preparation of di-p-nitrophenyl ester of dicarboxylic acids (2), and synthesis of PEAs (3) via solution polycondensation of (1) and (2).

(1) Synthesis of Di-p-toluenesulfonic Acid salt of Bis(L-arginine)-α,ω-Alkylene Diesters: L-arginine (0.02 mol) and 1,3-propanediol (0.01 mol) were refluxed in toluene (80 mL) in the presence of p-toluenesulfonic acid monohydrate (0.04 mol). The solid-liquid reaction mixture was heated to 120° C. and refluxed for 24 hr, generating 1.08 mL (0.06 mol) of water, which was collected in Dean-Stark reflux condenser. The reaction mixture (never completely dissolved) was then cooled to room temperature and toluene was decanted.

The dried reacted mixture was purified by re-precipitation twice from 2-propanol as follows. The mixture was placed in a 500 mL round bottom flask filled with 2-propanol, and refluxed at 100° C. until all the mixture was dissolved, then removed from heat, left in an oil bath overnight, and transferred to a freezer to form a white viscous mass as precipitate. The first re-precipitation yielded purified crystals, which were vacuum dried prior to the second re-precipitation. The product salt was a white powder obtained in nearly quantitative yield (˜90%).

(2) Synthesis of Di-p-nitrophenyl Ester of Dicarboxylic Acids: Di-p-nitrophenyl adipate (m.p. 123-124° C.) was prepared in nearly quantitative yield by the interaction of adipoyl chloride (1 mol) with p-nitrophenol (2.01 mol) in acetone in the presence of triethylamine (2.01 mol) at 0° to 5° C. The resulting di-p-nitrophenyl ester of adipic acid was purified by repeated recrystallization from acetonitrile.

(3) Solution condensation of (1) and (2): Products of (1) and (2) were added to 1.2 M dry N′N-dimethylacetamide solution in a reaction vessel and kept overnight without stirring at 65° C. in a thermostat controlled oven. The resulting viscous reaction solution was filtered through a glass filter and poured into distilled water. A tar-like mass of precipitate was thoroughly washed with distilled water for 3-7 days at room temperature to transform the tar-like substance into a non-sticky solid or rubbery polymer that still contained residual p-nitrophenol, a low molecular weight by-product of the solution polycondensation. To obtain the Arg-PEA free of p-nitrophenol, the polymer was precipitated from a methanol solution (10% v/w) into 15-20 fold excess (by volume) of ethylacetate. The precipitated polymer was separated by decanting the liquid phase, washed three to four times with fresh ethylacetate (40-50% of the starting volume of ethylacetate), and finally dried under a reduced pressure at 50-60 mm mercury to a constant weight. After drying, the polymer became corneous and was removed from the vessel by dissolving in chloroform and solvent casting onto glass plates. Obtained polymers were characterized by NMR spectroscopy and average molecular weights were determined by gel permeation chromatography (GPC).

### EXAMPLE 2

This example illustrates that cationic Arg-PEA shows low cytotoxicity and high efficiency when used as a gene transfer vector.

### A. Materials

PEI with a reported weight average molecular weight of 25 000, PLL-hydrobromide, ethidium bromide, MTT, phosphate-buffered saline (PBS, pH 7.4), HEPES, and DNA size markers were purchased from Sigma (St. Louis, Mo.) and SUPERFECT® was purchased from Qiagen (Valencia, Calif.). The pRL-CMV vector, a Dual-luciferase detection system, was obtained from Promega (Madison, Wis.). Other chemicals and reagents. if not otherwise specified, were purchased from Sigma (St. Louis, Mo.).

### B. Preparation of Plasmid DNA

Three luciferase encoding reporter plasmids, COL(-335)/LUC, COL(-772)/LUC, and pRL-CMV were provided by the laboratory of Dr. Bo Liu at Cornell Weill Medical College. All plasmids were prepared using endotoxin-free plasmid Maxi kits according to the supplier's protocol (Qiagen). The quantity and quality of the purified plasmid DNA was assessed by spectrophotometric analysis at 260 and 280 nm as well as by electrophoresis in 1% agarose gel. Purified plasmid DNAs were re-suspended in TE buffer and frozen in aliquots.

### C. Cell Culture

Rat aortic A10 vascular smooth muscle cells (SMC)s, obtained from American Tissue Culture Collection, were also kindly provided by the laboratory of Dr. Bo Liu at Cornell Weill Medical College. The SMCs were grown as recommended at 37° C. in 5% CO₂ in Dulbecco's minimal essential medium (DMEM) supplemented with 10% FBS (Germini, Woodland, Calif.) and antibiotics.

### D. Preparation of Complexes of DNA and Polymers for In Vitro Studies

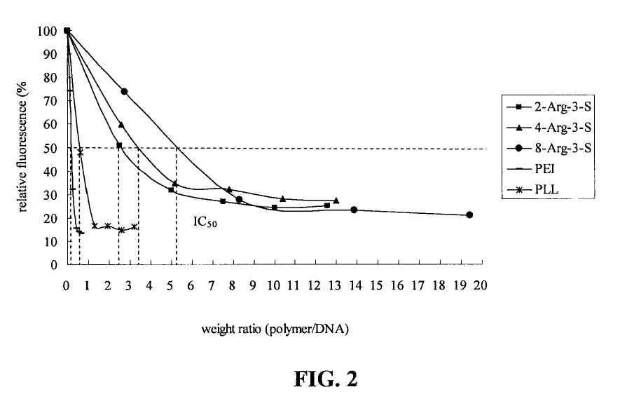

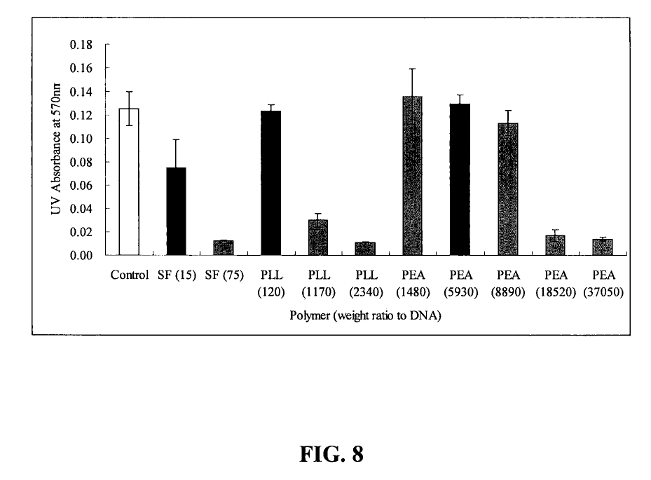

The cationic polymer/plasmid DNA complexes were prepared by adding aqueous solutions of the cationic Arg-PEA polymers to solution of the plasmid DNA in 20 mM HEPES buffer at pH 7.4, to obtain systems with specific DNA concentrations and polymer:DNA weight ratios as shown in FIGS. 2-9. The systems were immediately vortexed for several seconds after mixing the solutions, and then allowed to equilibrate at ambient conditions for 40 minutes. The ratio of polycation to DNA used to prepare complexes is represented as the weight ratio or the molar ratio of polycation nitrogen to DNA phosphate, i.e., the N:P ratio, at different circumstances. The stability of complexes in aqueous dispersion formed under these conditions was found to be strongly dependent on the buffer used. As a result, HEPES buffer was selected as solvent for the Arg-PEA polymers to afford greatest stability of polymer/DNA complexes.

### E. Gel Retardation Assay

Polymer/DNA complexes formed using the above protocol were analyzed by electrophoresis in a 1% agarose gel stained with ethidium bromide (EthBr) (10 μg/mL) with TAE buffer at 100 V for 60 min. DNA was visualized by UV illumination.

### F. Ethidium Bromide Displacement Assay

A standard ethidium bromide-DNA fluorescence displacement assay was performed to analyze the formation of polymer-DNA complexes. In a 96-well plate, each well was filled with 100 μL ethidium bromide solution (0.5 μg/mL), and background fluorescence (F_{bg}) with ethidium bromide alone was measured. Then 100 μL DNA solution (10 μg/mL calf thymus or plasmid DNA) was added and mixed well by pipetting up and down several times. Fluorescence of the DNA alone (F_{dna}) was measured at an excitation wavelength of 485/20 nm and an emission wavelength of 620/20 nm. All experiments were performed in triplicate.

Then aliquots of a test cationic polycation were added to the DNA solution, mixed gently by pipetting up and down, and fluorescence (Fₓ) was measured. The % inhibition of fluorescence (% Fᵢₙₕ) caused by presence of the cationic polymer was calculated by adding cationic polymer according to the following formula:

Fᵢₙₕ(%)=(Fₓ−F_{bg})/(F_{dna}−F_{bg})×100%

### G. Analysis of the Polymer/DNA Complexes by Gel Retardation Assay

In this experiment, a gel retardation assay was used to estimate the weight ratio of cationic polymer to DNA that results in a complete neutralization of the DNA. Complexes of plasmid DNA and 2-Arg-2-S cationic polymer were prepared at various weight ratios and analyzed by agarose gel electrophoresis as follows: 1.0 μg COL(-772)/LUC plasmid DNA only (lane 1), weight ratio of 2-Arg-2-S:DNA=4, 17, 42, 68 (lanes 2, 3, 4, 5, respectively). The aim was to quantify polyplex formation, the first and key step in non-viral gene therapy. DNA collapse, by charge neutralization of cationic polymers, is thought to be the key step in polyplex formation.

In a typical result obtained during electrophoresis experiments, movement of the plasmid in the gel was increasingly retarded as the amount of the 2-Arg-2-S PEA polymer increased, demonstrating that the 2-Arg-2-S PEA polymer above a certain weight ratio is able to bind to DNA and neutralize its charge. As the ratio of polymer to DNA increases, more gel retardation was observed (from lane 2 to lane 5). At weight ratio of Arg-PEA to DNA of 4:1 (lane 2), the complex moved only slightly toward the anode, indicating that a 4:1 weight ratio of 2-Arg-2-S was not enough to neutralize the DNA and the complex still possessed some negative charges. Complete neutralization was achieved at weight ratios from approximately 17:1 (lane 3) and higher. These results demonstrate the ability of cationic PEA, PEUR and PEU polymers to condense the plasmid DNA by neutralizing the charge on DNA, and provide the basic information for testing efficiency of a particular polymer as a gene carrier in a subsequent transfection experiment.

### H. Transient Transfection and Luciferase Assay

Complexes formed between plasmid DNA and the cationic polymers were assayed for their in vitro transfection activity utilizing a transient expression of luciferase reporter in smooth muscle cells (SMC A10). Transfection experiments were carried out according to the following protocol. SMC A10 cells were seeded at 30×10³ per well in a 24-well plate 24 hours before transfection to attain 70% confluent at transfection. One μg COL(-772)/LUC and pRL-CMV (10:1 weight ratio) was formulated with the different cationic polymers at various weight ratios. Briefly, for the L-Arg-PEA polymer, the transfection mixture was prepared as follows: 0.63 μl of DNA (1.595 μg/ul) and 6 to 600 μl of polymer (to obtain designated weight ratios) were added to an Eppendorf micro-tube containing 60 μl of DMEM. For the SUPERFECT® formulation, 1 μg of plasmid DNA in 60 μl serum-free DMEM were supplemented with 5 μl (3 μg/ul) of the SUPERFECT® solution in all experiments according to manufacturer's recommendation. The transfection mixtures were vortexed for a few seconds and then incubated at ambient conditions for 40 minutes. Immediately before transfection, cells were washed twice with PBS and DMEM, respectively, and then the transfection mixture plus 0.5 ml DMEM were added to each well. Cells were incubated in the wells for 3 h at 37° C. (5% CO₂), and then 0.5 ml of complete DMEM (10% fetal bovine serum (FBS), 1% Hepes, 1% penicillin-streptomycin) were added to each well and incubation was maintained. After 12 h, the medium in the wells was replaced with DMEM with 0.5% FBS. After another 24 hours, cells were harvested for luciferase assays.

Luciferase assays were performed according to the manufacturer's recommendation. Briefly, cells from each well of a 24-well plate were lysed in 100 μl lysis buffer, transferred to a micro-tube, and then centrifuged at 10 000 g for 2 min. Supernatants were collected and analyzed for luciferase activity. In a typical experiment 20 μl of supernatant was added to luminometric tubes containing 100 μl of luciferase substrate (Promega). Light emission was measured with a Dual-luciferase detection system for a period of 5 sec. while the relative light units were determined. Each experiment was performed in triplicate.

### I. Evaluation of Cytotoxicity of Gene Transfer Vectors

Cytotoxicity of the polymer/DNA complexes formed as described above was performed by MTT assay as follows. Cultured cells were seeded in 96-well plates at an appropriate cell density concentration (10 000 cells/well) and incubated overnight. Then the cells were treated with various polymer/DNA complex solutions. After 48 h of incubation, 15 μL of MTT solution (5 mg/mL) were added to each well, followed by incubation for three hours at 37° C., under 5% CO₂. The cell culture medium, including the complex-containing solution, was carefully removed and 150 μL of acidic isopropyl alcohol (with 0.1 M HCl) were added to dissolve the formed formazan crystal. Optical density (OD) was measured at 570 nm (subtract background reading at 690 nm) using a microplate reader. The percent cell viability (%) was calculated according to the following equation:

Viability(%)=(OD₅₇₀₍ₛₐₘₚₗₑ₎−OD₆₂₀₍ₛₐₘₚₗₑ₎)/(OD_{570(control)}−OD_{620(control)})×100%,

wherein OD_{570(control)} is the OD measurement from the wells treated with medium only and OD₅₇₀₍ₛₐₘₚₗₑ₎ is the reading from the wells treated with various polymer/plasmid DNA complexes.

### J. Results of Agarose Gel Retardation Study

To study the stability of various Arg-PEA polymer/DNA complexes and the time release of DNA from the polymer carrier under in vitro cell culture conditions, a series of polymer/DNA complexes were made as follows: 1.0 μg COL(-772)/LUC plasmid DNA only (lane 1), Arg-PEA polymers/DNA complexes at weight ratio of 100:2-Arg-3-S, (lane 2); 4-Arg-3-S, (lane 3) 8-Arg-3-S (lane 4); SUPERFECT® at weight ratio of 15 (optimum weight ratio recommended by manufacturer, lane 5); and polylysine (reported optimum weight ratio, lane 6). The complexes were incubated at 37° C. in a 5% CO₂ atmosphere for various periods of time (1 h, 6 h and 38 h) before an agarose gel electrophoresis assay was performed. The results of the assays were photographed. As was seen from the results of the gel retardation assay, after 1 hour of incubation, 8-Arg-3-S PEA polymer (lane 4) at weight ratio of 100:1 did not bind DNA or at least did not bind DNA longer than 1 hour. On the other hand, 2-Arg-3-S (lane 2) and 4-Arg-3 (lane 3) at weight ratio of 100:1 maintained binding with DNA during at least 6 hours of incubation. After 38 hours, the DNAs had been released from 4-Arg-3-S polymer, while DNAs remained bound to 2-Arg-3-S and migrated toward the anode of the assay. This result confirmed that, as the charge density of the Arg-PEA polymer decreases from 2-Arg-3-S to 4-Arg-3-S to 8-Arg-3-S (y=3, x=2, 4, 8 respectively), the ability of the polymer to bind and condense the DNA also decreases.

These results show, however, that strong binding and efficient DNA condensation do not correlate directly with gene-delivery efficiency, probably because tight binding prevents transcription. For optimal transfection efficiency, a gene transfer polymer must therefore balance sufficient binding strength to initially protect the plasmid with the ability to release the plasmid (See Pack, D. W et al. Design and development of polymers for gene transfer. Nature Reviews Drug Discovery (2005) 4:(7):581-593).

### K. Analysis of Polymer/DNA Complex by Ethidium Bromide Assays

The interaction between positively charged polymers and negatively charged DNA can also be demonstrated by an Ethidium Bromide assay. The assay is based on the well known principle that Ethidium bromide (EthBr) fluoresces intensely when intercalated into a DNA duplex. Fluorescence drops with breakdown of the DNA duplex and replacement of EthBr with polymer by condensation of the DNA with the polymer.

Therefore, increasing the amount of cationic polymer added to a given weight of DNA results in a reduction of EthBr fluorescence and corresponding breakdown of DNA duplex by condensation of DNA with polymer. The 50% inhibitory concentration (IC₅₀ value) corresponds to the polymer:DNA weight ratio required to produce 50% inhibition of fluorescence, and is used to compare the abilities of different cationic polymers to condense DNA. The chemical formula of each of the polymers tested is shown in Table in 2. Based on the chemical formulas of the polymers, it is shown that there is a positive charge center in every repeat unit of both PEI and PLL and there are about two positive charges in every repeat unit of each of the Arg-PEAs.

An EthBr assay was also conducted to compare the effect of the molecular weight of the polymer repeat unit on the positive charge density of a polymer. The results in the EthBr assay for PEI, PLL and various Arg-PEA polymers, as well as for various Arg-PEA polymers tested are shown in FIG. 2. It was determined that as the number of methylene groups in the repeat unit of the Arg-PEA increases, there is an increase in hydrophobicity of the Arg-PEA polymer. For example, 2-Arg-3-S reached the IC₅₀ value in the EthBr assay at a lower polymer:DNA weight ratio than did 4-Arg-3-S and 8-Arg-3-S. Therefore, the larger the molecular weight of the repeat unit, the less the positive charge density of the polymer.

When the value of x was fixed, and the value of y was varied in the chemical formula of the Arg-PEAs, an effect on DNA condensation rates was also illustrated in the EthBr assay: 2-Arg-2-S was compared with 2-Arg-3-S; 4-Arg-3-S with 4-Arg-4-S; and 8-Arg-3-S with 8-Arg-4-S. The results (FIG. 2) showed that 4-Arg-3-S condenses more rapidly than 4-Arg-4-S, and the IC₅₀ weight ratio difference was 16; whereas 8-Arg-3-S condenses DNA much faster than 8-Arg-4-S. Actually 8-Arg-4-S has a very weak ability to condense DNA as shown by a decrease in fluorescence of only 7% (i.e., to a value of 93%) even at the lowest weight ratio of 20:1. By contrast, 2-Arg-3-S reached 50% fluorescence reduction at a lower weight ratio than 2-Arg-2-S. These results illustrate the countervailing influence of steric hindrance on DNA condensation. When the value of y is small in the chemical formula, the two positively charged guanidino groups in the polymer unit are very close, hindering interaction of DNA with the guanidino groups, an effect that is more pronounced when y=2 than when y=3.

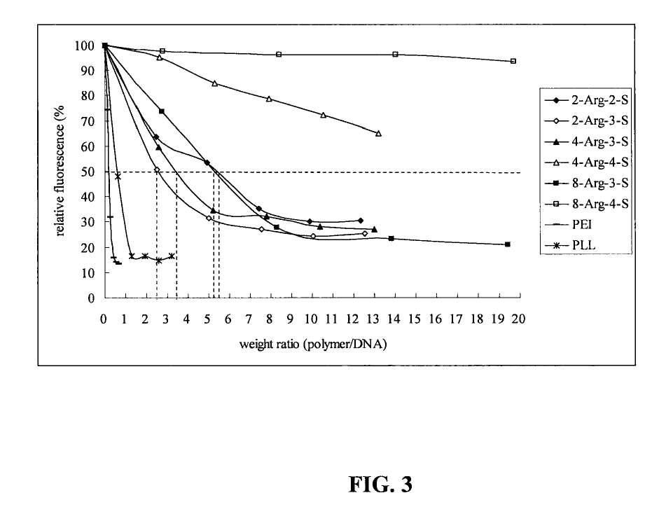

Based on these results, it can be concluded that when the repeat unit is short, or the charge density is high, steric hindrance plays a larger role than charge density in weakening the ability of cationic PEA, PEUR or PEU to condense DNA. As the repeat unit gets longer, or the space between two charged groups increases, the charge density plays a larger role (FIG. 3). The longer the length of the repeat unit, the larger is the difference, since with addition of a single methylene group (y changes from 3 to 4), the IC₅₀ weight difference between 8-Arg-3-S and 8-Arg-4-S is greater than that between 4-Arg-3-S and 4-Arg-4-S.

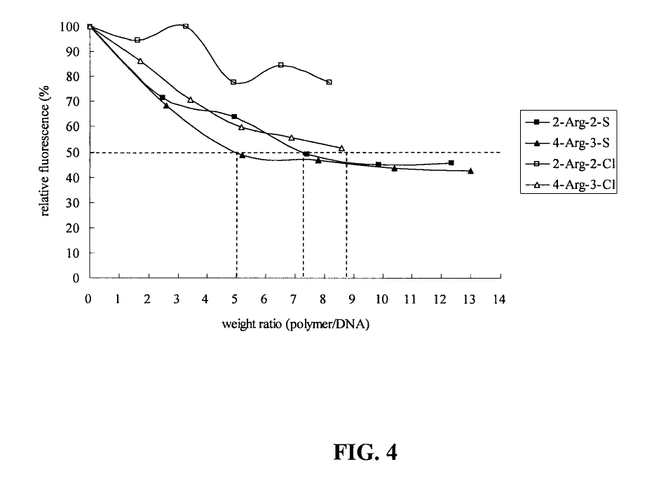

The EthBr assay was also used to illustrate the effect of different counter-ions on the ability of different Arg-PEAs to condense DNA. The two salts used for comparison were toluenesulfonic salt (counter-ion ═S) and hydrochloride salt (counter-ion=Cl). It was discovered (FIG. 4) that 4-Arg-3-S condensed DNA more efficiently than 4-Arg-3-Cl, and the IC₅₀ weight ratio difference was 4 weight ratio units; 2-Arg-2-S condensed DNA more efficiently than 2-Arg-2-Cl, and the difference was greater than 6 weight ratio units. The pKa of p-toluenesulfonic acid is −2.8, and the pKa of hydrochloric acid is −8, indicating that hydrochloric acid is a much stronger acid than p-toluenesulfonic acid. These results indicate that in a stronger acid, it is harder for DNA to compete with the acid to interact with the positively charged groups (e.g., guanidino) in the cationic PEA, PEUR and PEU polymers. Therefore, counter-ions from a weaker acid, for example having a pKa from about −7 to +5, are preferred to counter-ions from a stronger acid in the invention compositions.

In summary, these studies showed that increasing hydrophobicity or increasing the length between the charge centers in the polymer will decrease the efficiency of Arg-PEA polymer for condensing DNA, and the longer the length is between the charge centers, the greater is the decrease. But when the space between the two L-Arginine blocks is very small (y=2), steric hindrance starts to counteract the advantage produced by having a short distance between the charge centers in the polymer. Also, formation of a stronger counter-ion salt will decrease opportunities for DNA to interact with a positive center on a cationic Arg-PEA polymer and correspondingly decrease efficiency of the polymer for condensing DNA.

### L. Analysis of Transfection Efficiency Study

This experiment illustrates use of invention gene transfer compositions for in vitro transfection of vascular smooth muscle cells (SMCs) with plasmid DNA. Vascular SMCs were selected for testing the efficiency of arginine-based PEAs because vascular SMCs are the key to the formation of vascular lesions, which are major causes of stroke or infarction and also because vascular SMCs are very difficult to transfect with heretofore known non-viral gene vectors.

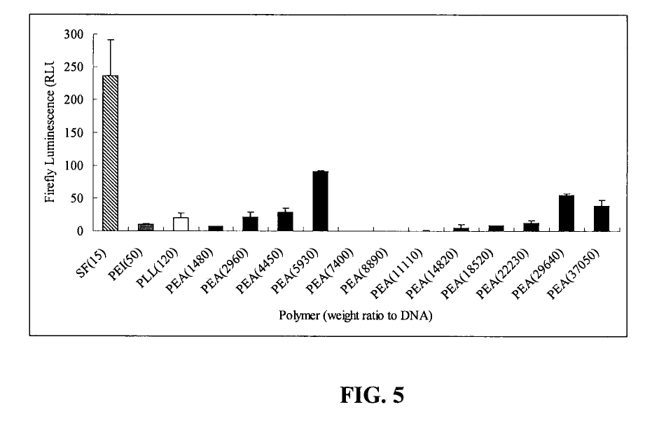

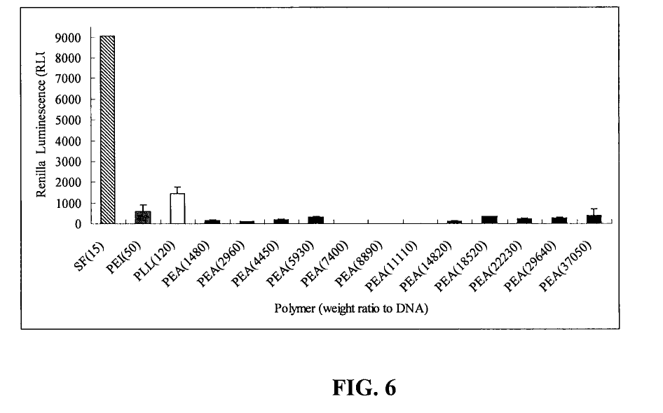

Two sets of plasmid DNA at a 10 to 1 weight ratio were used for this study, one encoding firefly luciferase driven by a collagen promoter, and the other encoding renilla luciferase driven by a CMV promoter. The results of these assays are shown in FIGS. 5 and 6. By measuring luciferase activities in cell lysates, which in this case is largely determined by the amount of DNA transferred into the cells, the transfection efficiency of Arg-PEAs was compared with that of SUPERFECT®, PEI and PLL. PEI and PLL polymers represent the earliest, the most reported upon and most efficient prior art non-viral transfection agents and provide a good reference point for evaluating the transfection efficiency of the invention gene transfer compositions for gene transfer. A popular commercial transfecting agent, SUPERFECT®, was also used in all experiments as a reference standard. The optimal transfection activity of PEI (25K Da) and PLL were observed at N/P ratio of approximately 4 and 2, respectively, a result which is consistent with the previous reports (Nguyen, H. K. et al. Gene Therapy (2000) 7(2):126-138 and D Oupicky et al. Stabilization of Polycation-DNA Complexes by Surface Modification with Hydrophilic Polymers, p. 61-78). Due to its superior condensing ability as shown in an EthBr displacement assay described above, the invention 2-Arg-3-S polymer was chosen out of a series of Arg-PEA polymers to illustrate transfection efficiency of the cationic PEAs, PEURs and PEUs in the invention gene transfer compositions.

The transfection efficiency of the various polymer:plasmid DNA complexes for successfully transforming vascular SMCs was tested using an EthBr displacement assay. FIG. 5 shows the firefly luciferase activity in cells that were transfected with various ratios of plasmid DNA:Arg-PEAs, PEI, PLL and SUPERFECT®. At the specified optimum conditions, luciferase activity, as a measure of transfection efficiency, increased with increased amount of 2-Arg-3-S up to 5927:1 weight ratio of polymer to DNA. Then efficiency dropped dramatically to almost zero activity comparable to that of naked DNA. When the weight ratio of 2-Arg-3-S PEA polymer to DNA was continuously increased from this point, the luciferase activity started to increase again from inactive to an activity comparable to that of PEI and PLL. However, at the highest ratios of Arg-PEA to DNA, cytotoxicity would have to be considered. Therefore, based on the results of this study, it was determined that optimum transfection activity of 2-Arg-3-S was found at the polymer:DNA weight ratio of 5927:1, which yielded transfection efficiency about 40% that of SUPERFECT®, and about 10-fold higher than that of PEI.

The weight ratio of 2-Arg-3-S to plasmid DNA required to reach optimum transfection efficiency was about 2000-fold higher than the weight ratio required to condense 50% of DNA, as indicated in the EthBr displacement assays described herein. This discovery indicates that the EthBr assay and gel retardation assay only demonstrate the amount of cationic polymer required to neutralize the negative charge of DNA, not necessarily an amount sufficient to carry condensed DNA through cell membranes and/or other barriers to transfection of cells. The high weight ratio requirement of Arg-PEAs polymers for successful transfection of vascular SMCs probably can be attributed to two factors: 1) low charge density of Arg-PEAs (for example, compare the two charge centers per 814.92 MW of Arg-PEA with one charge center per 43 MW of PEI), and 2) blocking of positive charges of Arg-PEA by pre-occupying p-toluenesulfonic acid. Since the Arg-PEA polymers have very good water solubility and are biodegradable, the only possible concern for using such a great amount of the polymer is possible cytotoxicity of the polymers to cells.

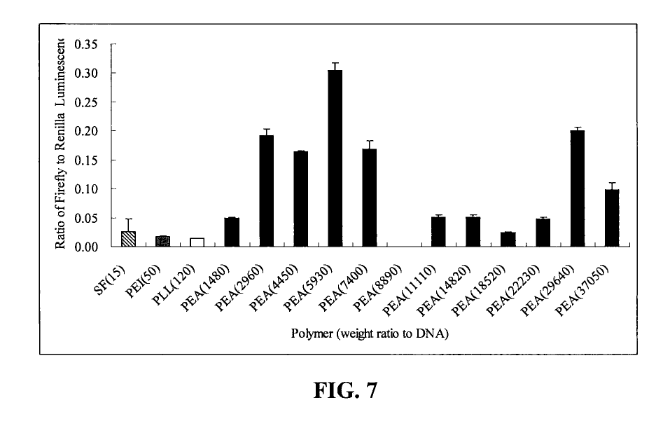

Interestingly, the activity of renilla luciferase plasmid co-transfected into vascular SMCs was found to be lower in cells transfected using an Arg-PEA:plasmid complex than in control polymers (FIG. 6). Since the amount of renilla plasmid DNA used in the assay was only one tenth that of the firefly DNA, it is possible the Arg-PEA has selective affinity for DNA above a certain concentration. On the other hand, when the ratio of firefly luciferase activity is compared to that of renilla luciferase as shown in FIG. 7, the highest relative luciferase activity ratio of PEAs was about 10-fold higher than that of SUPERFECT®, PEI and PLL, a result that indicates Arg-PEA polymers deliver DNAs with greater uniformity during co-transfection than the known delivery systems tested.

### M. Analysis of Cytotoxicity of Polymers by MTT Assay

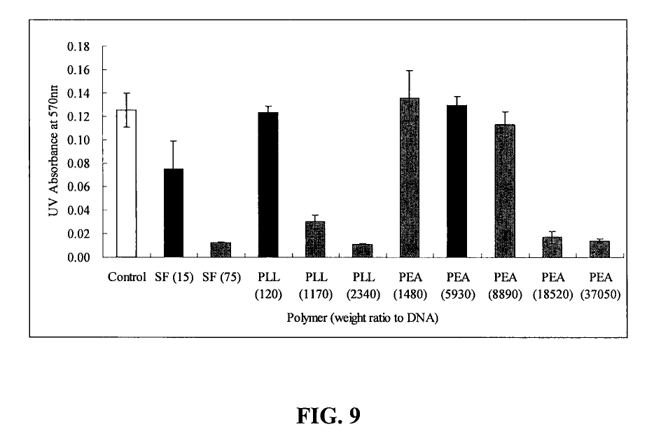

Cytotoxicity of polymers was evaluated by MTT assay, a simple, accurate, reproducible means of detecting living cells via mitochondrial dehydrogenase activity. In this assay, an increase in cell number (cell proliferation) results in an increase in the amount of MTT formazan formed and results in an increase in UV absorbance of this compound. Since PLL is well known to be a much less toxic transfection agent than PEI, PLL was tested as a control to compare with Arg-PEA polymers. As shown by the results of the cytotoxicity assay summarized in FIG. 9, the dose of SUPERFECT® needed to reach optimum transfection efficiency, although very little compared with that of the optimum Arg-PEA (2-Arg-3-S PEA), still led to a reduction in cell viability of about 40%. Similar cytotoxicity was seen with high doses of PLL. In contrast, cells transfected with Arg-PEA at a high range of polymer:DNA weight ratio exhibited cell viability comparable to that of PLL at its optimum weight ratio up to an extreme high weight ratio, such as 18520:1. These results showed that high charge density in a gene transfer polymer is highly correlated to high cytotoxicity. Since the cationic PEAs have a much lower positive charge density than other gene transfer polymers, a much larger weight ratio of cationic PEA is needed to achieve efficient transfection, but cationic PEA at such a high weight ratio does not adversely affect the viability of transfected cells.

### N. Image Analysis of Polymer/DNA Complex

To visually confirm the transfection efficiency reading obtained from the luciferase activity tests, SMCs were transfected with plasmid DNAs encoding Green Fluorescent Protein (GFP). Two days following transfection, the cells were examined under a fluorescence microscope for their expression of GFP (cells turn green). The results of observation showed that GFP plasmid DNAs were successfully expressed by PEA polymer, the same as commercial product SUPERFECT®®. Cytotoxicity of the polymers was studied by observing morphology of the GFP expressing cells under a light microscope and by the results of a MTT assay. SMCs treated with 15 μg or 1500 μg of 2-Arg-2-S PEA for 24 hours displayed normal smooth muscle cell morphology, confirming the low cytotoxicity of the Arg-PEA polymers. In contrast, SMCs that were incubated with 15 μg of PEI or SUPERFECT® appeared to be deformed in different degrees as seen under the light microscope.

Although the invention has been described with reference to the above examples, it will be understood that modifications and variations are encompassed within the spirit and scope of the invention. Accordingly, the invention is limited only by the following claims.

What is claimed is:

1. A composition comprising at least one of the following polymers: a PEA polymer having a chemical formula described by general structural formula (I), 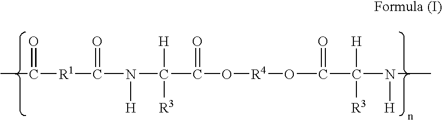
wherein n ranges from about 5 to about 100; R¹ is independently selected from (C₂-C₅) alkyl or alkenyl; R³s in individual n units are independently selected from the group consisting of (CH₂)₃NHC(═NH₂⁺)NH₂, 4-methylene imidazolinium, (CH₂)₄NH₃⁺, (CH₂)₃NH₃⁺ and combinations thereof; and R⁴ is independently (C₂-C₅) alkyl;
or a poly(ester urethane) (PEUR) polymer having a chemical formula described by structural formula (II), 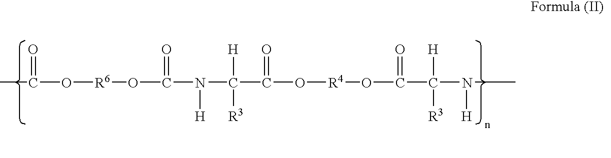
wherein n ranges from about 5 to about 100; R³s in individual n units are independently selected from the group consisting of (CH₂)₃NHC(═NH₂⁺)NH₂, (CH₂)₄NH₃⁺, (CH₂)₃NH₃⁺, 4-methylene imidazolinium, and combinations thereof; and R⁴ and R⁶ are independently (C₂-C₅) alkyl;
or a PEU having a chemical formula described by general structural formula (III): 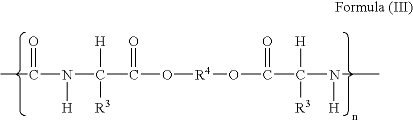
wherein n ranges from about 5 to about 100; R³s in individual n units are independently selected from the group consisting of (CH₂)₃NHC(═NH₂⁺)NH₂, (CH₂)₄NH₃⁺, (CH₂)₃NH₃⁺, 4-methylene imidazolinium, and combinations thereof; and
R⁴ is independently (C₂-C₅) alkyl.

2. The composition of claim 1 further comprising a nucleic acid, wherein the composition has a weight ratio of the polymer to the nucleic sufficient to substantially neutralize electrical charge of the composition.

3. The composition of claim 1, wherein the R³s comprise (CH₂)₄NH₃⁺.

4. The composition of claim 1, wherein the R³s comprise (CH₂)₃NHC(═NH₂⁺)NH₂).

5. The composition of claim 1, wherein the R³s comprise 4-methylene imidazolinium ion.

6. The composition of claim 1, further comprising at least one counter-ion from a weak acid associated with the polymer.

7. The composition of claim 2, wherein the poly nucleic acid comprises a gene encoding a therapeutic polypeptide.

8. The composition of claim 2, wherein the poly nucleic acid comprises RNA.

9. The composition of claim 8, wherein the RNA comprises antisense poly nucleic acid that is complimentary to an mRNA that encodes the target protein.

10. The composition of claim 2, wherein the polymer:poly nucleic acid weight ratio is about 6000:1 to about 5000.1.

11. A method for transfecting a target cell comprising:
contacting a target cell with a composition of claim 2 under conditions suitable to transfect the target cell with the poly nucleic acid.

12. The method of claim 11, wherein the R³s comprise (CH₂)₄NH₃⁺).

13. The method of claim 11, wherein the R³s comprise (CH₂)₃NHC(═NH₂⁺)NH₂).

14. The method of claim 11, wherein the R³s comprise:

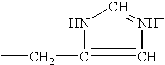

15. The method of claim 11, wherein the poly nucleic acid comprises a gene encoding a therapeutic polypeptide.

16. The method of claim 15, wherein the poly nucleic acid further comprises plasmid DNA suitable for expressing the gene in the target cell.

17. The method of claim 16, wherein the target cell is associated with a disease.

18. The method of claim 11, wherein the poly nucleic acid comprises RNA.

19. The method of claim 18, wherein the RNA comprises antisense poly nucleic acid that is complimentary to an mRNA that encodes a target protein.

20. The method of claim 18, wherein the poly nucleic acid comprises iRNA for suppression of a target gene in the target cell.

21. The method of claim 20, wherein the iRNA forms siRNA.
